(19) United States
(12) Patent Application Publication
(10) Pub. No.: US 2025/0072748 A1
(43) Pub. Date: Mar. 6, 2025

(54) SYSTEMS AND METHODS FOR SPATIALLY MAPPING CORNEAL BIOMECHANICAL PROPERTIES

(71) Applicant: Alcon Inc., Fribourg (CH)

(72) Inventors: Gangjun LIU, Portland, OR (US); Lingfeng YU, Rancho Santa Margarita, CA (US)

(21) Appl. No.: 18/815,135

(22) Filed: Aug. 26, 2024

Related U.S. Application Data
(60) Provisional application No. 63/579,392, filed on Aug. 29, 2023.

(51) Int. Cl. A61B 3/10; A61B 5/00

(52) U.S. Cl. CPC A61B 3/102; A61B 3/1015; A61B 5/0053

(57) ABSTRACT

Systems and methods for measuring corneal biomechanical properties are provided. In certain embodiments, a method comprises determining a plurality of optical coherence tomography (OCT) measurement locations in a region of a cornea; for each OCT measurement location: applying a mechanical excitation to the cornea at the OCT measurement location, and measuring a response of the cornea to the mechanical excitation by acquiring temporal OCT data at the OCT measurement location; generating temporal deformation data for the region of the cornea based on the temporal OCT data at each OCT measurement location; and generating biomechanical data for the region of the cornea based on the temporal deformation data.

### CROSS-REFERENCE TO RELATED APPLICATIONS

This application claims the benefit of U.S. Provisional Application Ser. No. 63/579,392 (filed on Aug. 29, 2023), the content of which is incorporated herein by reference in its entirety.

### BACKGROUND

The present disclosure relates to measuring corneal biomechanical properties. More particularly, the present disclosure relates to an optical coherence tomography (OCT) system for spatially mapping corneal biomechanical properties.

In a medical imaging context, an OCT system directs a coherent light beam towards biological tissue, and then measures the interference between a portion of the original coherent light beam and the scattered light reflected back to the OCT system from a particular location on (or within) the biological tissue. The interference is directly related to the reflectivity of the biological tissue at that location. The OCT system generates a one-dimensional “A-scan” by measuring the reflectivity at different depths (axial dimension) at the same location using time domain OCT (TD-OCT) or frequency (Fourier) domain OCT (FD-OCT). The OCT system generates a two-dimensional “B-scan” by combining A-scans acquired at different lateral locations (lateral dimension). For example, an ocular or ophthalmic OCT system may be used to acquire high-resolution images of the retina, the cornea, the ocular lens, and the iris to determine ocular dimensions, to diagnose various ocular pathologies, etc.

The cornea has complex viscoelastic biomechanical properties, which means that the cornea exhibits both viscous and elastic behavior when deformed, such as in response to a mechanical stimulus. Concomitantly, the biomechanical properties of the cornea are non-uniform because the cornea is comprised of a non-uniform material, which means that different regions of the cornea have different biomechanical properties. Unfortunately, none of the current corneal biomechanical measurement techniques provide spatially-resolved corneal biomechanical information.

### SUMMARY

Embodiments of the present disclosure advantageously provide systems and methods for measuring corneal biomechanical properties. In certain embodiments, a method comprises determining a plurality of OCT measurement locations in a region of a cornea. For each OCT measurement location, the method further comprises applying a mechanical excitation to the cornea at the OCT measurement location, and measuring a response of the cornea to the mechanical excitation by acquiring temporal OCT data at the OCT measurement location. The method further comprises generating temporal deformation data for the region of the cornea based on the temporal OCT data at each OCT measurement location. In addition, the method comprises generating biomechanical data for the region of the cornea based on the temporal deformation data.

### BRIEF DESCRIPTION OF THE DRAWINGS

FIG. 1 depicts a block diagram of an example OCT system for measuring corneal biomechanics, in accordance with embodiments of the present disclosure.

FIG. 2 depicts a block diagram of an example OCT engine, in accordance with embodiments of the present disclosure.

FIG. 3A depicts a block diagram of an example control computer, in accordance with embodiments of the present disclosure.

FIG. 3B depicts a block diagram of another example control computer, in accordance with embodiments of the present disclosure.

FIG. 4 depicts a graph illustrating corneal deformation versus time, in accordance with embodiments of the present disclosure.

FIG. 5 depicts a flow diagram illustrating functionality associated with measuring corneal biomechanics, in accordance with embodiments of the present disclosure.

### DETAILED DESCRIPTION

The measurement of corneal biomechanics will significantly impact the ophthalmic industry by enabling, inter alia, quicker and more proficient diagnosis of corneal disease, safer and more effective surgical treatments, the provision of personized care, etc. More particularly, spatially mapping the biomechanical properties of the whole cornea will be important for the early diagnosis of disease such as glaucoma, keratoconus, etc.

Embodiments of the present disclosure advantageously measure biomechanical properties at a number of locations within one or more regions of the cornea to provide a spatial map of the biomechanical properties of the cornea. Additionally, embodiments of the present disclosure may identify strong and weak portions or regions of the cornea, measure large deformations as well as micrometer to nanometer range deformations, and provide sub-micron resolution that quantifies the biomechanical properties of a diseased or healthy cornea thereby providing new metrics for treatment planning.

Furthermore, spatially mapping the biomechanical properties of the cornea improves the predictability of treating surgery-induced astigmatism (SIA) at the time of cataract surgery, identifies patients at risk for complications post-LASIK, and improves the predictability of corneal treatments. Moreover, spatially mapping the biomechanical properties of the cornea may be integrated into treatment regimens to produce more accurate outcomes, such as cataract outcomes from patient-specific screenings, assessments, and interventions, outcomes for limbal relaxing incisions (LRI) based on patient-specific calculations, outcomes for orthokeratology (ortho-k), treatment decisions for corneal refractive Px, etc.

To wit, embodiments of the present disclosure advantageously provide systems and methods for measuring corneal biomechanical properties. In certain embodiments, a method comprises determining a plurality of OCT measurement locations in a region of a cornea. For each OCT measurement location, the method further comprises applying a mechanical excitation to the cornea at the OCT measurement location, and measuring a response of the cornea to the mechanical excitation by acquiring temporal OCT data at the OCT measurement location. The method further comprises generating temporal deformation data for the region of the cornea based on the temporal OCT data at each OCT measurement location. In addition, the method comprises generating biomechanical data for the region of the cornea based on the temporal deformation data.

The biomechanical properties or data at each OCT measurement location in the region may comprise, for example, the largest deformation amplitude for a given mechanical excitation, hysteresis, elastic modulus (or Young's modulus), shear modulus, natural frequency, etc.

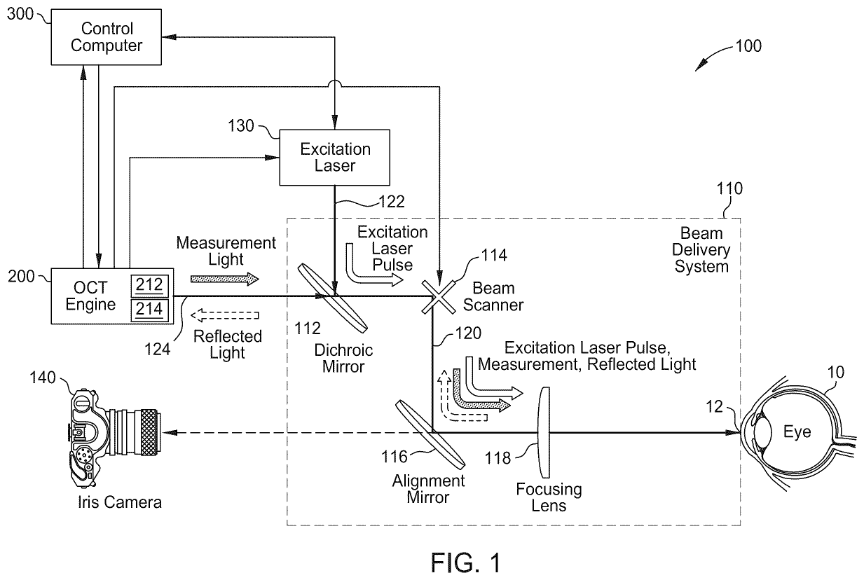

FIG. 1 depicts a block diagram of OCT system 100 for measuring corneal biomechanics, in accordance with embodiments of the present disclosure.

In certain embodiments, OCT system 100 includes, inter alia, beam delivery system (BDS) 110, excitation laser 130, OCT engine 200 and control computer 300. In certain embodiments, iris camera 140 may be provided to align eye 10 and cornea 12 to BDS 110.

In certain embodiments, OCT engine 200 and control computer 300 are separate devices that are communicatively coupled using a wired or wireless connection, such as USB, Ethernet, Bluetooth, WiFi, etc. In certain other embodiments, OCT engine 200 is a component of control computer 300, such as one or more peripheral component interconnect express (PCIe) expansion boards, a PCIe expansion board coupled to one or more external modules, etc. In certain embodiments, OCT engine 200 may be housed within an external PCIe expansion system enclosure coupled to control computer 300 via a PCIe connection.

Generally, BDS 110 is configured to, inter alia, receive, propagate and focus excitation laser pulses from excitation laser 130 onto cornea 12, to receive, propagate and focus low-coherence light from OCT engine 200 onto cornea 12, and to receive, focus, propagate and direct reflected light from cornea 12 to OCT engine 200.

In certain embodiments, BDS 110 is a free-space optical system that includes dichroic mirror 112, beam scanner 114, alignment mirror 116, and focusing lens 118 that define common optical path 120. Excitation laser pulses from excitation laser 130 propagate along optical path 122 to BDS 110, light from low-coherence light source 212 of OCT engine 200 propagates along optical path 124 to BDS 110, and reflected light from BDS 110 propagates along optical path 124 to reflected light detector 214 of OCT engine 200.

Dichroic mirror 112 is configured to reflect (or deflect) the excitation laser pulses propagating along optical path 122 into common optical path 120, to pass the light propagating along optical path 124 into common optical path 120, and to pass the reflected light propagating along common optical path 120 into optical path 124.

Beam scanner 114 is configured to adjust the OCT measurement location on cornea 12 in two dimensions, such as the lateral dimension and the elevation dimension, in response to commands received from OCT engine 200. Advantageously, beam scanner 114 scans the excitation laser pulses and the light from low-coherence light source 212 together, while common optical path 120 ensures that the excitation laser pulses and the light from low-coherence light source 212 travel coaxially and focus on the same location on cornea 12 in order to properly align the excitation and detection components of OCT system 100. Generally, beam scanner 114 reflects (or deflects) excitation laser pulses, low-coherence light, and the reflected light along common optical path 120.

Beam scanner 114 may include a single mirror that rotates about two orthogonal axes to adjust the OCT measurement location on cornea 12 in two dimensions. Alternatively, beam scanner 114 may include two orthogonal mirrors, and each mirror independently rotates about an orthogonal axis to adjust the OCT measurement location on cornea 12 in two dimensions. The single mirror may be driven by a pair of electric motors or galvanometers (or galvos), while each orthogonal mirror may be driven by a single electric motor or galvanometer. Other drive systems are also contemplated, such as piezoelectric actuators, piezoelectric galvanometers, magnetorestrictive actuators, etc.

Alignment mirror 116 is configured to mechanically align cornea 12 to common optical path 120 of BDS 110. Generally alignment mirror 116 reflects (or deflects) excitation laser pulses, low-coherence light, and the reflected light 90° along common optical path 120. In certain embodiments, alignment mirror 116 passes the image of cornea 12 to iris camera 140 to align cornea 12 to common optical path 120. In certain embodiments, OCT system 100 does not include alignment mirror 116, and common optical path 120 includes a straight optical path segment between beam scanner 114 and focusing lens 118.

Focusing lens 118 focuses the excitation laser beams and the low-coherence light traveling along common optical path 120 onto cornea 12, and focuses the reflected light from cornea 12 into common optical path 120.

In certain other embodiments, BDS 110 may be a fiber-based system in which certain free-space optical elements are replaced by optical fibers, electro-optical elements, etc. For example, optical fiber may replace at least certain portions of the free-space optical paths, such as portions of common optical path 120, optical path 122 and optical path 124, a fiber optic-based beam combiner may replace dichroic mirror 112, an electro-optical beam steering chip with an optical phased array (OPA) may replace beam scanner 114, etc.

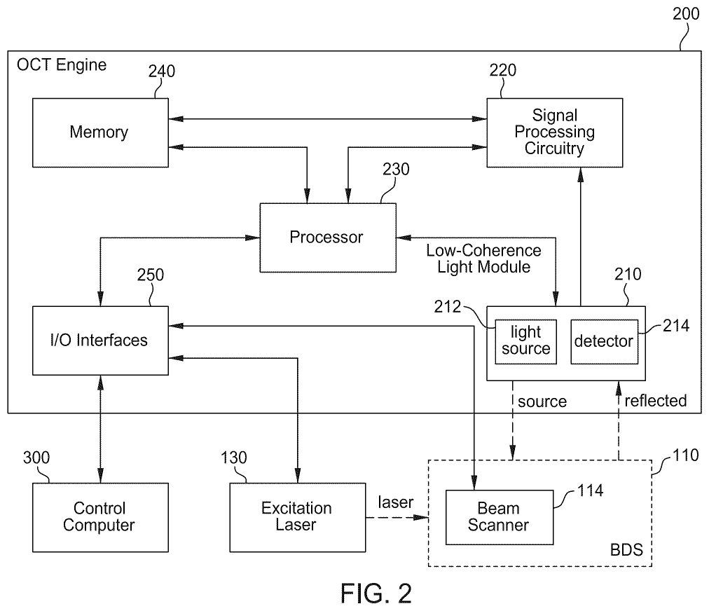

FIG. 2 depicts a block diagram of OCT engine 200, in accordance with embodiments of the present disclosure.

In certain embodiments, OCT engine 200 includes, inter alia, low-coherence light module 210, signal processing circuitry 220, one or more processor(s) 230, storage element or memory 240, and I/O interfaces 250. Low-coherence light module 210 includes low-coherence light source 212 and reflected light detector 214. Signal processing circuitry 220 may be coupled to low-coherence light module 210 and memory 240, and processor 230 may be coupled to low-coherence light module 210, signal processing circuitry 220, memory 240, and I/O interfaces 250. In certain embodiments, signal processing circuitry 220 is not present, and the functionality provided by signal processing circuitry 220 is provided by processor 230.

In certain embodiments, OCT engine 200 provides FD-OCT measurements using swept-source OCT (SS-OCT). In certain SS-OCT embodiments, low-coherence light source 212 may be a swept-wavelength light source and reflected light detector 214 may be a high-speed photodetector. The swept-wavelength light source is configured to rapidly sweep a narrow line-width optical signal over a broad range of wavelengths during each A-scan, such as a Fourier Domain Mode Locked (FDML) laser with a 1050 nm center wavelength and a scan rate of 5 MHz or greater. Generally, the sweep rate of the swept-wavelength light source may be 100 kHz or greater. The high-speed photodetector is configured to sequentially detect the wavelength components of the reflected light signal (or interferometric signal) during one wavelength sweep (A-scan). In other words, reflected light detector 214 is configured to produce a spectral interferogram with fringe patterns during each wavelength sweep (A-scan). The spectral interferogram includes intensity data for each wavelength (or frequency) emitted by the swept-wavelength light source.

In other embodiments, OCT engine 200 provides FD-OCT measurements using spectral domain (SD-OCT). In certain SD-OCT embodiments, low-coherence light source 212 may be a broadband optical source such as a superluminescent diode (SLD), and reflected light detector 214 may include a spectrometer and a high speed line camera that generates the spectral interferogram with fringe patterns during each wavelength sweep (A-scan).

A high-speed analog-to-digital (A/D) converter may be coupled to, or included within, low-coherence light module 210 to convert the analog interferometric signal into a digital signal, which is generally known as OCT data. In certain embodiments, the high-speed A/D converter may convert the analog interferogram signals into 12-bit digital words at rates of 150 MSPS (or greater) with a 1 GHz bandwidth (or greater).

In certain embodiments, signal processing circuitry 220 is coupled to low-coherence light module 210, and includes one or more microprocessors, application specific integrated circuits (ASICs), field programmable gate arrays (FPGAs), etc., configured to generate complex OCT data based on the OCT data received from reflected light detector 214. In certain embodiments, signal processing circuitry 220 includes a high-speed A/D converter (rather than low-coherence light module 210), which converts the analog spectral interferogram signals received from reflected light detector 214 into OCT data.

Advantageously, the OCT data simultaneously includes intensity information for all of the depth layers in the A-scan at the OCT measurement location.

In certain embodiments, signal processing circuitry 220 may extract the intensity information for each depth layer in the A-scan by applying wavenumber remapping, dispersion compensation, Fourier transformation, etc., to the OCT data to generate complex OCT data. For example, signal processing circuitry 220 may apply a fast Fourier transform (FFT) to the OCT data to generate the complex OCT data. The amplitude of the complex OCT data may be squared to yield the intensity at different depths, while the phase of the complex OCT data may be further processed to provide deformation information, as described below.

During a motion mode scan (also known as an M-mode scan), OCT engine 200 performs multiple A-scans at the same location, which generates temporal OCT data for that location. And, signal processing circuitry 220 may apply an FFT to the temporal OCT data to generate temporal complex OCT data for that location.

Processor 230 may include one or more general-purpose or application-specific microprocessors, microcontrollers, etc., that execute instructions to perform control, computation, input/output, etc. functions for OCT engine 200. For example, processor 230 may be configured to synchronize the triggering of excitation laser 130, the scanning of beam scanner 114, and the measurement operations of low-coherence light module 210 in order to acquire and send OCT data and complex OCT data to control computer 300.

Processor 230 may include a single integrated circuit, such as a micro-processing device, or multiple integrated circuit devices and/or circuit boards working in cooperation to accomplish the appropriate functionality. In certain embodiments, signal processing circuitry 220 is not present, and processor 230 may apply wavenumber remapping, dispersion compensation, Fourier transformation (such as an FFT), etc., to the OCT data to generate complex OCT data.

Generally, memory 240 may store instructions for execution by processor 230 as well as data, such as OCT data, complex OCT data, etc. Memory 240 may include a variety of non-transitory computer-readable medium that may be accessed by processor 230 as well as other components. In various embodiments, memory 240 may include volatile and nonvolatile medium, non-removable medium and/or removable medium. For example, memory 240 may include any combination of random access memory (RAM), dynamic RAM (DRAM), static RAM (SRAM), read only memory (ROM), flash memory, cache memory, and/or any other type of non-transitory computer-readable medium.

I/O interfaces 250 are configured to transmit and/or receive data from control computer 300, such as OCT measurement locations and complex OCT data, send commands to excitation laser 130 to emit laser excitation pulses, send commands to beam scanner 114 to adjust the focus point of common optical path 120 to different OCT measurement locations on cornea 12, etc. Generally, data may be sent over wired and/or wireless connections. For example, I/O interfaces 370 may include one or more wired communications interfaces, such as USB, Ethernet, etc., and/or one or more wireless communications interfaces, coupled to one or more antennas, such as Bluetooth, WiFi, etc.

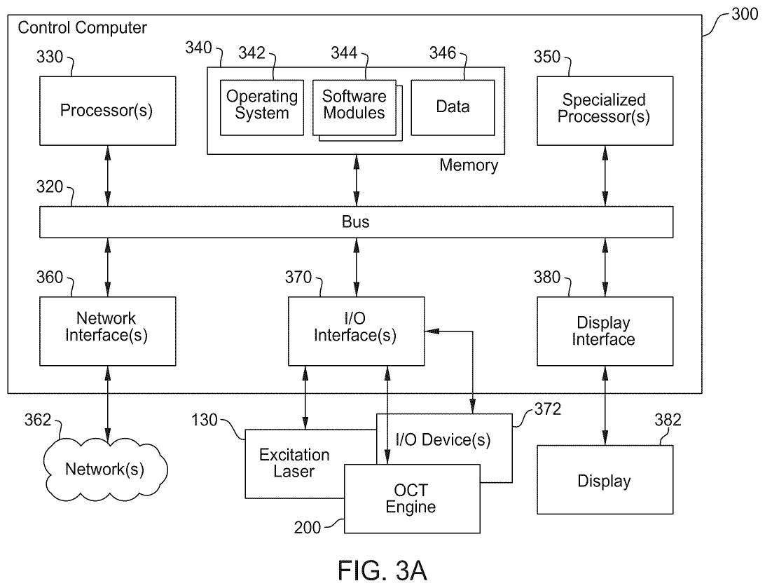

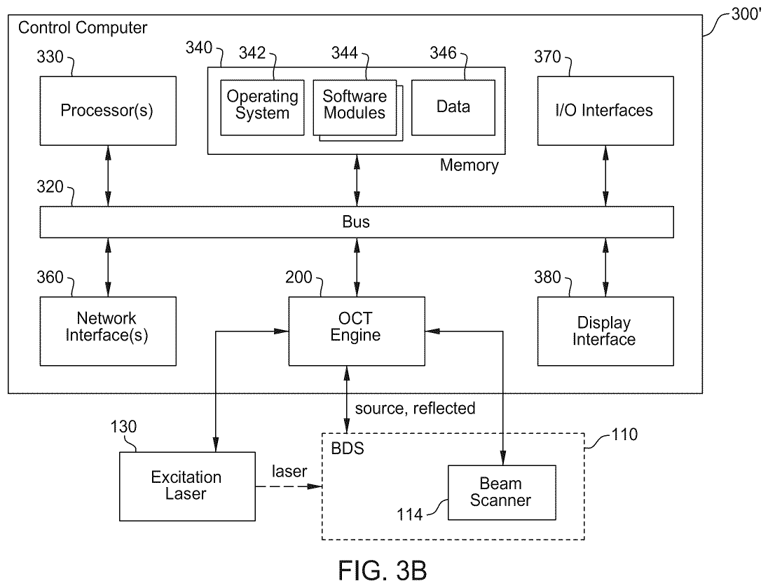

FIG. 3A depicts a block diagram of control computer 300, in accordance with embodiments of the present disclosure.

Control computer 300 includes bus 320 coupled to one or more processor(s) 330, storage element or memory 340, one or more network interface(s) 360, I/O interfaces 370, and display interface 380. In certain embodiments, control computer 300 also includes one or more specialized processor(s) or processing circuitry 350, such as, for example, graphics processing units (GPUs), ASICs, FPGAs, etc. Generally, network interface(s) 360 are coupled to one or more network(s) 362 using a wired or wireless connection, I/O interfaces 370 are coupled to one or more I/O device(s) 372 using a wired or wireless connection, and display interface 380 is typically coupled to display 382 using a wired connection.

In certain embodiments, excitation laser 130 and OCT engine 200 are coupled to I/O interfaces 370 using a wired or wireless connection, such as USB, Ethernet, Bluetooth, WiFi, etc. In certain embodiments, excitation laser 130 may send status information to control computer 300, over the connection, and control computer 300 may send configuration information, power commands, etc., to excitation laser 130 over the connection. Here, a laser may be used to introduce the mechanical movement. Other methods such as air puff, ultrasound or mechanical vibration may also be used to introduce the stimulation of the cornea.

Bus 320 is a high-speed data transfer subsystem, such as a PCIe bus, etc., that transfers data between processor 330, memory 340, network interface(s) 360, I/O interfaces 370, and display interface 380. In certain embodiments, bus 320 also transfers data between these components and specialized processor or processing circuitry 350.

Processor 330 includes one or more general-purpose or application-specific microprocessors that execute instructions to perform control, computation, input/output, etc. functions for control computer 300. Each processor 330 may include a single integrated circuit, such as a micro-processing device, or multiple integrated circuit devices and/or circuit boards working in cooperation to accomplish the appropriate functionality. In addition, processor 330 may execute computer programs or modules, such as operating system 342, software modules 344, etc., stored within memory 340.

Generally, memory 340 stores instructions for execution by processor 330 as well as data. Memory 340 may include a variety of non-transitory computer-readable medium that may be accessed by processor 330 as well as other components. In various embodiments, memory 340 may include volatile and nonvolatile medium, non-removable medium and/or removable medium. For example, memory 340 may include any combination of random access memory (RAM), dynamic RAM (DRAM), static RAM (SRAM), read only memory (ROM), flash memory, cache memory, and/or any other type of non-transitory computer-readable medium.

Memory 340 stores various components for retrieving, presenting, modifying, and storing data, such as operating system 342, software modules 344, etc. Operating system 342 provides operating system functionality for control computer 300, while software modules 344 provide certain functionality when executed by processor 330. Data 346 may include data associated with operating system 342, software modules 344, etc.

Network interface(s) 360 is configured to transmit data to and from network(s) 362 using a wired and/or wireless connection. For example, network(s) 362 may include a local area network (LAN) that is connected to a wide area network (WAN) through a router, the WAN may be connected to the Internet through an Internet Service Provider (ISP), etc. Network(s) 362 may execute various network protocols, such as, for example, wired and/or wireless Ethernet, Bluetooth, etc. Network(s) 362 may also include various combinations of wired and/or wireless physical layers, such as, for example, copper wire or coaxial cable networks, fiber optic networks, WiFi networks, Bluetooth mesh networks, CDMA, FDMA and TDMA cellular networks, etc.

I/O interfaces 370 are configured to transmit and/or receive data from I/O devices 372. I/O interfaces 370 enable connectivity between processor 330, memory 340 and I/O devices 372 by encoding data to be sent from processor 330 or memory 340 to I/O devices 372, and decoding data received from I/O devices 372 for processor 330 or memory 340. Generally, data may be sent over wired and/or wireless connections. For example, I/O interfaces 370 may include one or more wired communications interfaces, such as USB, Ethernet, etc., and/or one or more wireless communications interfaces, coupled to one or more antennas, such as Bluetooth, WiFi, etc.

Generally, I/O devices 372 provide input to control computer 300 and/or output from control computer 300. As discussed above, I/O devices 372 are operably connected to control computer 300 using a wired and/or wireless connection. I/O devices 372 may include a local processor coupled to a communication interface that is configured to communicate with control computer 300 using the wired and/or wireless connection. For example, I/O devices 372 may include a touch screen, keyboard, mouse, touch pad, joystick, etc.

Display interface 380 is configured to transmit image data from control computer 300 to monitor or display 382.

FIG. 3B depicts a block diagram of control computer 300′, in accordance with embodiments of the present disclosure.

Control computer 300′ includes bus 320 coupled to one or more processor(s) 330, storage element or memory 340, one or more network interface(s) 360, I/O interfaces 370, and display interface 380, as described above with respect to control computer 300. In FIG. 3B, OCT engine 200 is depicted as a component of control computer 300′, such as one or more PCIe expansion boards, a PCIe expansion board coupled to one or more external modules, etc. In certain embodiments, OCT engine 200 may be housed within an external PCIe expansion system enclosure coupled to bus 320 via a PCIe connection.

The regions of interest for the measurement may depend on the purpose of the measurement. For diagnosis purposes, a large measurement area should be covered. The measurement locations may be uniformly distributed around the whole cornea. For surgery preparation purposes, such as cataract surgery, etc., the regions of interest may be the location(s) where the corneal incision will be performed. M-mode is the preferred scanning mechanism for the measurement. The measurement procedure for each location includes stimulation laser excitation following by M-mode imaging for a continuous time, such as 5 milliseconds, 8 milliseconds, etc. Then, control computer 300 may command beam scanner 114 to move the laser beam to another location and repeat the excitation and M-mode imaging at the allocation. The excitation and M-mode imaging procedure will repeat for each location until all the locations are finished.

In certain embodiments, excitation laser 130 mechanically excites or stimulates cornea 12 by emitting at least one laser beam pulse which generates a micro-deformation that produces a shear wave that radiates from the OCT measurement location. Because a single micro-deformation is desired to be generated for each OCT measurement, control computer 300 adjusts the energy of the pulsed laser beam emitted by excitation laser 130 to ensure that the correct amount of mechanical excitation is imparted to cornea 12, as well as to ensure patient safety.

Starting with the first OCT measurement location in the scan pattern, OCT engine 200 triggers excitation laser 130, which will emit one (or a few pulses) that mechanically excite (deform) the surface of cornea 12 at the OCT measurement location. After mechanical excitation, OCT engine 200 performs an M-mode scan or a BM-mode scan at the OCT measurement location, which generates temporal OCT data for that location. Signal processing circuitry 220 may then apply an FFT to the temporal OCT data to generate temporal complex OCT data for that location. OCT engine 200 then commands beam scanner 114 to move to the next OCT measurement location in the scan pattern until temporal OCT data and temporal complex OCT data for all of the OCT measurement locations in the scan pattern have been acquired.

Generally, processor 330 (or specialized processor 350) may be configured to determine the largest deformation amplitude and the hysteresis at each OCT measurement location by processing the OCT data generated by OCT engine 200. Because the deformation is acquired with using M-mode (or BM-mode) scanning, and because the excitation laser pulses and the light from low-coherence light source 212 are co-axial, the temporal OCT data will always capture the largest deformation location.

A typical OCT has an axial resolution of a few microns. In order to detect submicron or nanometer level deformation, in many embodiments, phase resolved method such as a Doppler OCT algorithm may be applied to detect the deformation. In a typical Fourier domain OCT system, the data processing may involve the Fourier transform or Fast Fourier Transform (FFT) on the acquired spectrum fringes to get the depth coded complex value tissue reflectance. The amplitude of the depth encoded complex value tissue reflectance will be displayed as an OCT image. In a phase resolved method, such as Doppler OCT, etc., the phase of the depth-coded complex value tissue reflectance is further processed. To obtain the submicron or nanometer level deformation, the phase difference between the complex reflectance signal acquired at different times from the same location are calculated. This phase difference are converted to deformation. In an M-mode scanning situation, all the A-lines are acquired from the same location and these A-lines represent temporal reflectance signals from the same location. The 1^{st} A-line will be the baseline reflectance signal, and the rest of the A-line signals will be compared to the baseline signal to obtain the temporal change of the signal. The temporal changes represents the corneal deformation under external excitation, and will be used for the analysis. In a BM-mode scanning situation, repeated B-Mode scanning passes are performed at the same location. Comparison is then performed between the baseline B-scan signal (typically the 1^{st} B-scan) and the rest of the B-scan signals.

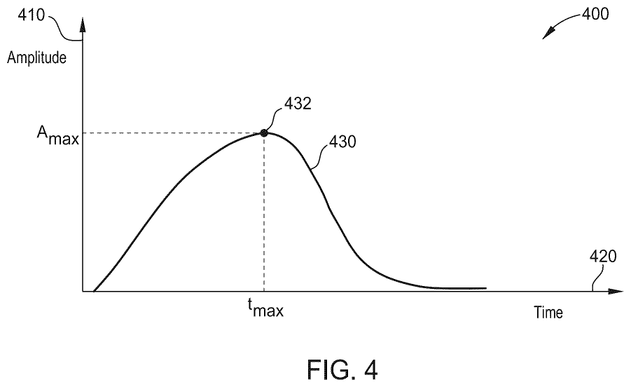

FIG. 4 depicts graph 400 illustrating corneal deformation versus time, in accordance with embodiments of the present disclosure.

Graph 400 illustrates corneal deformation curve 430 plotted with respect to amplitude axis 410 vs. time axis 420. In certain embodiments, corneal deformation curve 430 may be provided by phase resolved methods, such as the Doppler OCT algorithm, etc. The biomechanical properties, such as largest deformation amplitude 432, hysteresis, etc., may be obtained from corneal deformation curve 430.

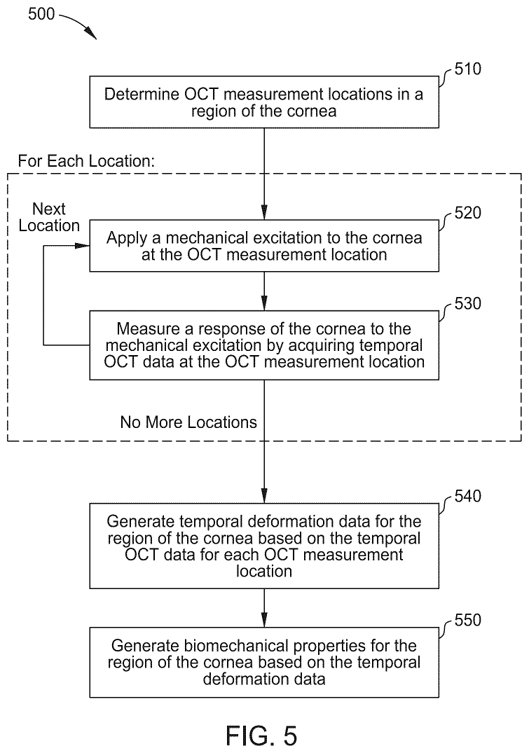

FIG. 5 depicts flow diagram 500 illustrating functionality associated with measuring corneal biomechanics, in accordance with embodiments of the present disclosure.

At 510, a plurality of OCT measurement locations in a region of cornea 12 are determined. Block 510 may be performed, for example, by control computer 300 or 300′. As discussed above, control computer 300 or 300′ may divide cornea 12 into a number of regions, and then divide each region into a number of OCT measurement locations which form a two-dimensional scan pattern for the region, such as a symmetric or asymmetric scan pattern, a square or rectangular scan pattern, a circular or oval scan pattern, etc. Flow continues to block 520.

Blocks 520 and 530 are performed sequentially for each OCT measurement location in the region. In other words, blocks 520 and 530 are performed at the first OCT measurement location in the scan pattern for the region, blocks 520 and 530 are then performed at the second OCT measurement location in the scan pattern for the region, and so on until temporal OCT data have been acquired at all of the OCT measurement locations in the scan pattern for the region.

At 520, a mechanical excitation to cornea 12 at the OCT measurement location is applied. Block 520 may be performed, for example, by excitation laser 130. As described above, excitation laser 130 may emit one or more excitation laser pulses that propagate along optical path 122 and common optical path 120 to the OCT measurement location. Flow continues to block 530.

At 530, a response of cornea 12 to the mechanical excitation is measured by acquiring temporal OCT data at the OCT measurement location. Block 530 may be performed, for example, by OCT engine 200, as described above. If there are additional OCT measurement locations in the scan pattern for the region, flow returns to block 520 for the next OCT measurement location; otherwise, flow continues to block 540.

At 540, temporal deformation data for the region of cornea 12 is generated based on the temporal OCT data at each OCT measurement location for the region. Block 540 may be performed, for example, by control computer 300 or 300′, as described above. Flow continues to block 550.

At 550, biomechanical data for the region of cornea 12 is generated based on the temporal deformation data. Block 550 may be performed, for example, by control computer 300 or 300′. As discussed above, the biomechanical properties or data generated at each OCT measurement location in the region may include, for example, the largest deformation amplitude for a given mechanical excitation, hysteresis, etc.

In certain other embodiments, a non-optical mechanical excitation device, such as an air puff generator, an ultrasonic pulse generator, a physical actuator, etc., mounted on a two-dimensional positioning table, may be used in place of excitation laser 130. The two-dimensional positioning table may be used to coordinate the mechanical scanning of the non-optical mechanical excitation device with the scanning of the OCT measurement locations provided by beam scanner 114.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A method for measuring corneal biomechanical properties, the method comprising:
determining a plurality of optical coherence tomography (OCT) measurement locations in a region of a cornea;
for each OCT measurement location:
applying a mechanical excitation to the cornea at the OCT measurement location, and
measuring a response of the cornea to the mechanical excitation by acquiring temporal OCT data at the OCT measurement location;
generating temporal deformation data for the region of the cornea based on the temporal OCT data at each OCT measurement location; and
generating biomechanical data for the region of the cornea based on the temporal deformation data.

2. The method of claim 1, wherein the biomechanical data for the region of the cornea comprise:
a largest deformation amplitude at each OCT measurement location; and
a hysteresis at each OCT measurement location;

3. The method of claim 2, wherein the biomechanical data for the region of the cornea further comprise:
a natural frequency at each OCT measurement location.

4. The method of claim 2, wherein:
the acquiring temporal OCT data comprises:
acquiring M-mode OCT data, and
generating complex OCT data based on the M-mode OCT data; and
the generating temporal deformation data for the region of the cornea comprises:
generating relative phase difference data between adjacent OCT measurement locations based on the complex OCT data, and
generating temporal deformation data for each OCT measurement location based on the relative phase difference data.

5. The method of claim 4, wherein the generating the complex OCT data comprises processing the M-mode OCT data based on a wavenumber remapping, a dispersion compensation, or a fast Fourier transform (FFT).

6. The method of claim 2, wherein:
the applying the mechanical excitation to the cornea at the OCT measurement location comprises emitting, by an excitation laser, one or more excitation laser pulses that propagate along a common optical path to the OCT measurement location; and
a beam delivery system (BDS) defines the common optical path.

7. The method of claim 6, wherein the acquiring temporal OCT data comprises:
emitting, by a low-coherence light source, light that propagates along the common optical path to the OCT measurement location;
detecting, by a reflected light detector, reflected light that propagates along the common optical path from the OCT measurement location; and
generating, by a processor or signal processing circuitry coupled to the reflected light detector, the temporal OCT data based on the reflected light.

8. The method of claim 7, wherein the BDS comprises a dichroic mirror, a beam scanner, an alignment mirror, and a focusing lens that define the common optical path.

9. The method of claim 8, wherein the dichroic mirror is configured to:
reflect the excitation laser pulses from the excitation laser into the common optical path;
pass the light from the low-coherence light source into the common optical path; and
pass the reflected light from the common optical path to the reflected light detector.

10. The method of claim 9, wherein the excitation laser pulses and the light propagate coaxially along the common optical path to the OCT measurement location.

11. The method of claim 1, wherein the applying the mechanical excitation comprises:
directing an air puff to the OCT measurement location; or
directing one or more ultrasonic pulses to the OCT measurement location;

12. The method of claim 1, further comprising:
dividing the cornea into a plurality of regions;
measuring biomechanical properties of the cornea in each region; and
determining, based on the biomechanical properties of each region the cornea, a spatial map of the biomechanical properties of the cornea.

13. A system for measuring corneal biomechanical properties, the system comprising:
a beam delivery system (BDS) defining a common optical path;
an excitation laser;
an optical coherence tomography (OCT) engine configured to:
send a command to the excitation laser to apply a mechanical excitation to a cornea at an OCT measurement location via the common optical path, and
measure a response of the cornea to the mechanical excitation by acquiring temporal OCT data at the OCT measurement location via the common optical path; and
a control computer, coupled to the OCT engine, the control computer comprising a processor configured to:
determine a plurality of OCT measurement locations in a region of the cornea,
send the OCT measurement locations to the OCT engine,
receive temporal OCT data for each OCT measurement location from the OCT engine,
generate temporal deformation data for the region of the cornea based on the temporal OCT data for each OCT measurement location, and
generate biomechanical data for the region of the cornea based on the temporal deformation data.

14. The system of claim 13, wherein the biomechanical data for the region of the cornea comprise:
a largest deformation amplitude at each OCT measurement location; and
a hysteresis at each OCT measurement location;

15. The system of claim 14, wherein:
the OCT engine comprises a low-coherence light source, a reflected light detector, and a processor or signal processing circuitry configured to generate the temporal OCT data;
the excitation laser is configured to emit one or more excitation laser pulses that propagate along the common optical path to the OCT measurement location;
the low-coherence light source is configured to emit light that propagates along the common optical path to the OCT measurement location; and
the reflected light detector is configured to detect reflected light that propagates along the common optical path from the OCT measurement location.

16. The system of claim 15, wherein the BDS comprises a dichroic mirror, a beam scanner, an alignment mirror, and a focusing lens that define the common optical path.

17. The system of claim 16, wherein the dichroic mirror is configured to:
reflect the excitation laser pulses from the excitation laser into the common optical path;
pass the light from the low-coherence light source into the common optical path; and
pass the reflected light from the common optical path to the reflected light detector.

18. The system of claim 17, wherein the excitation laser pulses and the light propagate coaxially along the common optical path to the OCT measurement location.

19. The system of claim 14, wherein
the temporal OCT data comprise complex OCT data that are based on M-mode OCT data; and
generate temporal deformation data for the region of the cornea comprises:
generate relative phase difference data between adjacent OCT measurement locations based on the complex OCT data, and
generate temporal deformation data for each OCT measurement location based on the relative phase difference data.

20. The system of claim 19, wherein generate the complex OCT data comprises process the M-mode OCT data based on a wavenumber remapping, a dispersion compensation, or a fast Fourier transform (FFT).
